(19) United States
(12) Patent Application Publication
(10) Pub. No.: US 2012/0157500 A1
(43) Pub. Date: Jun. 21, 2012

(54) JAK INHIBITION BLOCKS RNA INTERFERENCE ASSOCIATED TOXICITIES

(76) Inventor: Weikang Tao, Lansdale, PA (US)

(21) Appl. No.: 13/391,393

(22) PCT Filed: Aug. 16, 2010
(86) PCT No.: PCT/US2010/045618

§ 371 Date: Feb. 21, 2012

Related U.S. Application Data
(60) Provisional application No. 61/236,238, filed on Aug. 24, 2009.

(51) Int. Cl. A61K 31/4439; A61K 31/4412; A61K 31/165

(52) U.S. Cl. 514/341; 514/609; 514/345

(57) ABSTRACT

The instant invention provides a method for treating patients by administering a JAK inhibitor. The instant invention provides a method for treating patients by administering a JAK inhibitor wherein the JAK inhibitor is a JAK2 inhibitor. The instant invention provides a method for treating patients by administering a JAK inhibitor wherein the JAK inhibitor is selected from selected from Jak2-IA, AG490, Pyridone 6, WP1066, LS104, TG101209, TG101348, CP690,550, CP352,664, INCB18424, WHI-P154, CMP6, SB1518, XL019, CEP-701, INCB20, AUH-6-96 and AZ960.

### BACKGROUND OF THE INVENTION

Synthetic small interfering RNA (siRNA) duplexes hold a great promise to become a new therapeutic entity as they are able to silence gene expression specifically in a sequence-dependent manner by triggering RNA interference (RNAi), an evolutionarily conserved cellular process for repressing gene expression^{1, 2}. Given the fact that naked siRNAs, even with optimized sequences and chemical modifications, lack drug-like pharmacokinetic properties, tissue bioavailability and the ability of entering cells, a major hurdle for harnessing siRNA for broad therapeutic use is the effective and safe delivery of siRNA to the diseased tissues and cells via systemic administration^{3, 4}. Many platforms such as liposomes, lipoplexes, antibody and cholesterol conjugates, and cationic polymers, have been developed for systemic delivery of siRNA^{5, 6}. Among these, cationic liposome-based vehicles are the most widely validated means for liver delivery and have demonstrated a superior activity in delivering siRNA to hepatocytes in rodents and non-human primates (NHP), resulting in a robust target gene knockdown and the mechanism-based pharmacological sequela⁷⁻¹⁰. Recently several liposome-assembled siRNA drugs have entered clinical trials for an evaluation of their pharmacokinetic and pharmacodynamic properties and safety profiles.

One major concern on the approach of using cationic lipid-based carriers for systemic delivery of siRNA is the potential to trigger the innate immune response, anaphylactic reaction, liver damage and other systemic toxicities independently of target gene repression^{4, 11}, since cationic liposome-assembled DNA plasmid or antisense oligonucleotides elicited such toxic responses^{12, 13}. Recently, it was shown that intravenous administration of cationic lipid-encapsulated siRNA nanoparticles stimulated the innate immune system, leading to an induction of proinflammatory cytokines and serum transaminases in mice and NHP^{7, 14-16}, which resembles the toxicities observed with a cationic lipid and plasmid DNA assemblies¹². Although the scope and magnitude of toxic responses may vary depending on liposomal compositions and the nature of nucleic acid payloads, activation of innate immunity characterized by cytokine/chemokine induction is commonly seen among liposomal siRNA-triggered reactions^{4, 11, 17}.

The innate immune system consists of membrane-associated Toll-like receptors (TLRs), cytoplasmic RNA-binding immunoreceptors and the receptor-linked signaling pathways¹⁷⁻²⁰. While TLRs located at the plasma membrane, such as TLR-2 and TLR-4, function to recognize nonself lipid components, TLRs residing at endosomal membrane including TLR-3, TLR-718 and TLR-9 as well as cytoplasmic RNA sensors are responsible for detecting foreign nucleic acids through the recognition of specific molecular patterns. Ligand-stimulated TLRs or cytoplasmic sensors elicit cytokine induction via activating the IKK/NFkB, p38/AP1, IRF3/5/7 (interferon (IFN) regulatory factor) and PI3K pathways^{18, 21-24}. Induced cytokines further stimulate the production and secretion of cytokines/chemokines and drive inflammatory response by engaging the JAK/STAT and NFkB pathways^{21, 25-27}. The JAK/STAT pathway which is associated with receptors of multiple cytokines is essential for executing inflammation/immune responses. Overstimulation of the innate immune system is pathologic^{11, 28, 29}. Liposome-formulated siRNA nanoparticles have the potential to stimulate both lipid- and RNA-sensing TLRs, as well as cytoplasmic immunoreceptors. Although sequence optimization and chemical modifications of siRNA are effective in lowering siRNA-mediated TLR-stimulating activity^{16, 30}, it is unclear whether these procedures can eradicate the immunostimulatory property of siRNA in vivo. In addition to immunostimulation, lipid-mediated interaction and cytotoxicity may directly damage blood cells, endothelial cells and hepatocytes resulting in a secondary inflammation and multi-systemic toxicities. For an effective management of toxic responses to liposomal siRNA-based therapeutics and an aid in the development of safer delivery vehicles, it is important to elucidate the mechanism underlying liposomal siRNA toxicities.

### BRIEF DESCRIPTION OF THE FIGURES

FIG. 1: Lipid composition, in vivo target silencing activities and toxicities of LF01-SSB and LF01-ApoB nanoparticles.

(a) Lipid structure and composition of the LF01 liposome.

(b) LF01-SSB and LF01-ApoB silenced target gene specifically. Rats (4 per group) were dosed with vehicle (PBS), LF01-SSB or LF01-ApoB at 1 mg/kg via tail vein injection. 24 hr later, mRNA levels of SSB, ApoB and Ppib (a housekeeping gene) in liver medial lobe were determined by qRT-PCR. The quantities of SSB and ApoB mRNA relative to Ppib levels are presented. Bars indicate standard error of means (SEM).

(c) Overview of the experimental protocol for evaluating toxicities in rats.

(d) Summary of LF01-SSB-induced toxicities in rats. As depicted in (c), after an IV dose of PBS or LF0-SSB, rats (5 per group) were monitored for lethality and red urine. Animals died or having red urine in each group were scored. Blood and tissue samples were collected at different time points for various analyses as indicated (c). Data are presented as the mean±SEM (n=5). All animals receiving 9 mg/kg of LF01-SSB survived through 3 hr but died by 24 hr. For this group, only 3 hr cytokine data were collected. ND: not done.

FIG. 2: Chemical structure and pharmacokinetic and pharmacodynamic properties of Jak2-IA.

(a) Chemical structure of Jak2-IA.

(b) Pharmacokinetic and pharmacodynamic properties of Jak2-IA in C57B1/6 mice. Mice (4 per group) were co-dosed with aranesp to activate Jak2, and either Jak2-IA or vehicle. Blood was collected at 1, 3 and 8 hrs post dosing and analyzed for Jak-2-mediated phosphorylation of STAT5 (p-STAT5) in blood cells and Jak2-IA concentrations in plasma. The levels of p-STAT5 as a function of time and Jak2-IA doses are presented. Plasma concentrations of Jak2-IA are also shown. Data are shown as the mean±SEM.

(c) Pharmacokinetic property of Jak2-IA in Sprague-Dawley (SD) rats. 2 rats were dosed with Jak2-IA (100 mg/kg, p.o.) and blood was collected at indicated times for evaluation of plasma concentrations of Jak2-IA. The average of measurements from 2 rats was shown.

FIG. 3: Pretreatment with Jak2-IA or dexamethasone abrogates LF01-SSB-induced toxicities in rats.

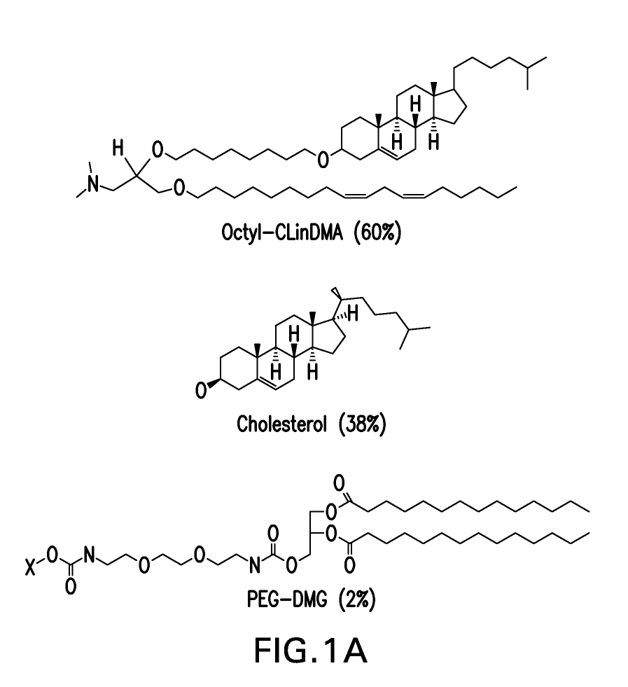

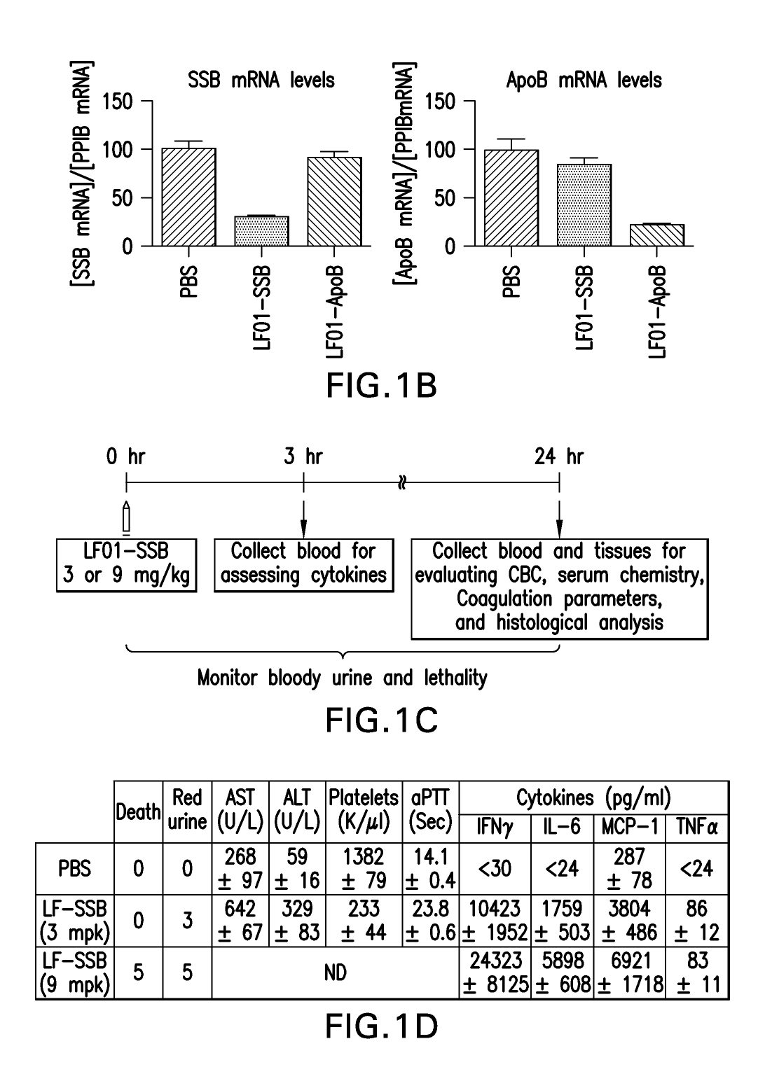

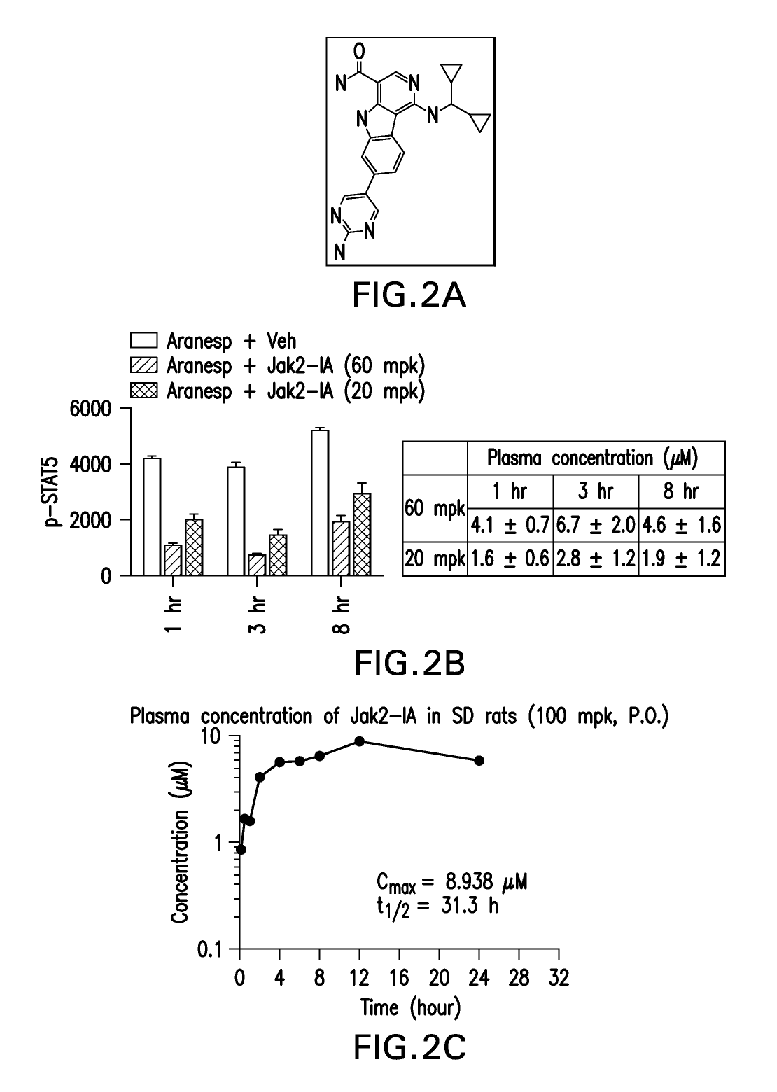

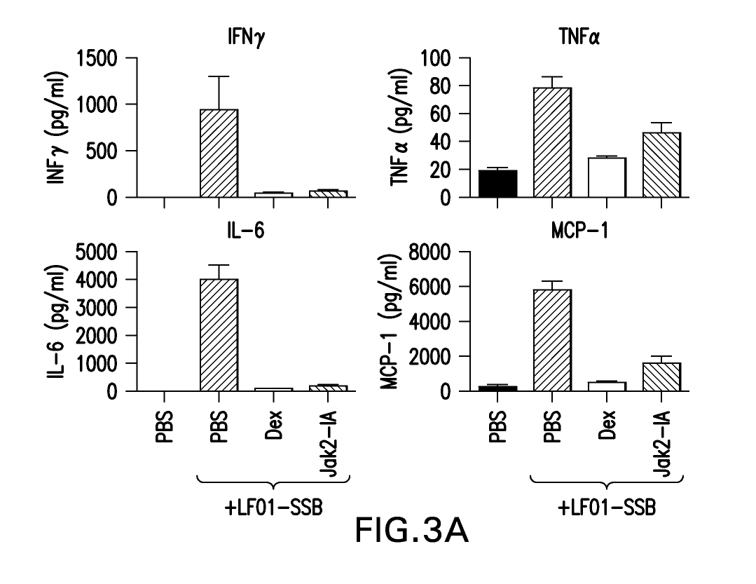

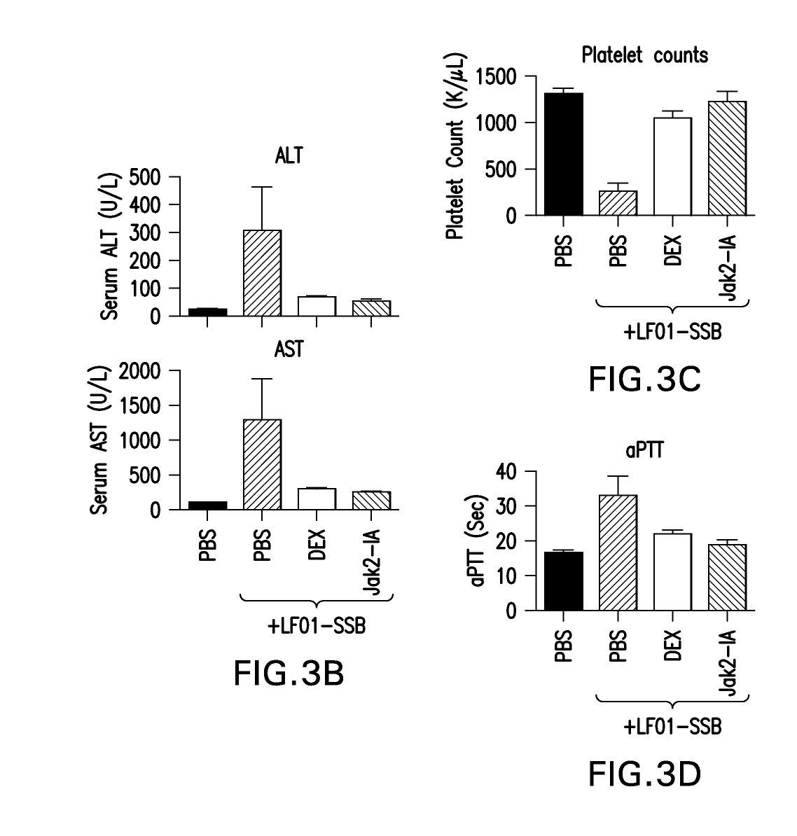

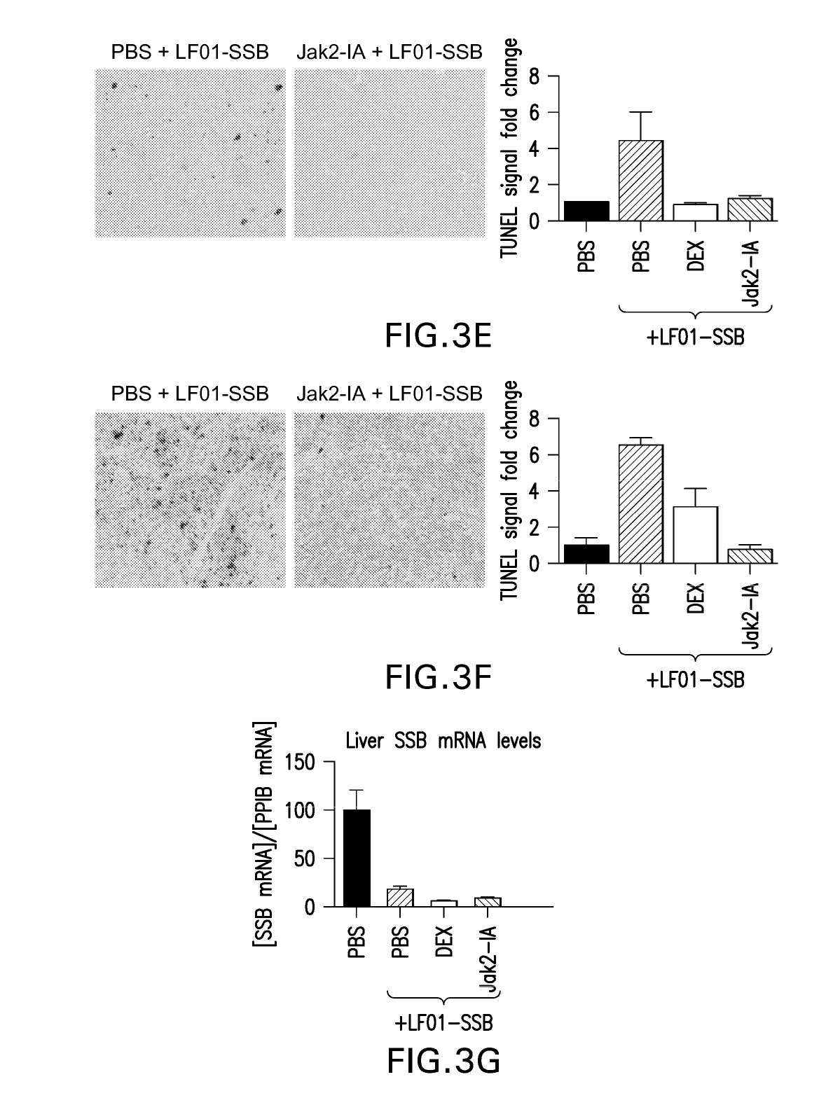

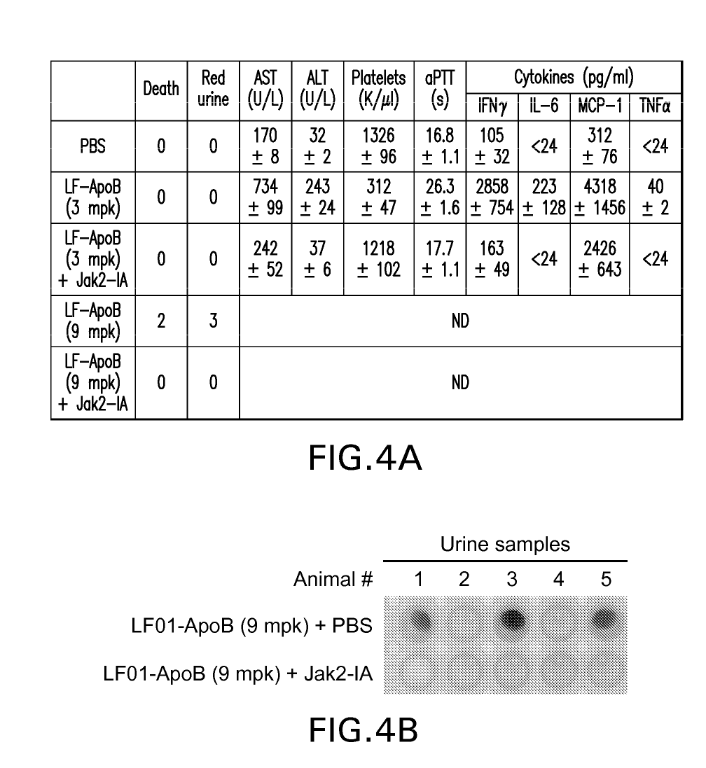

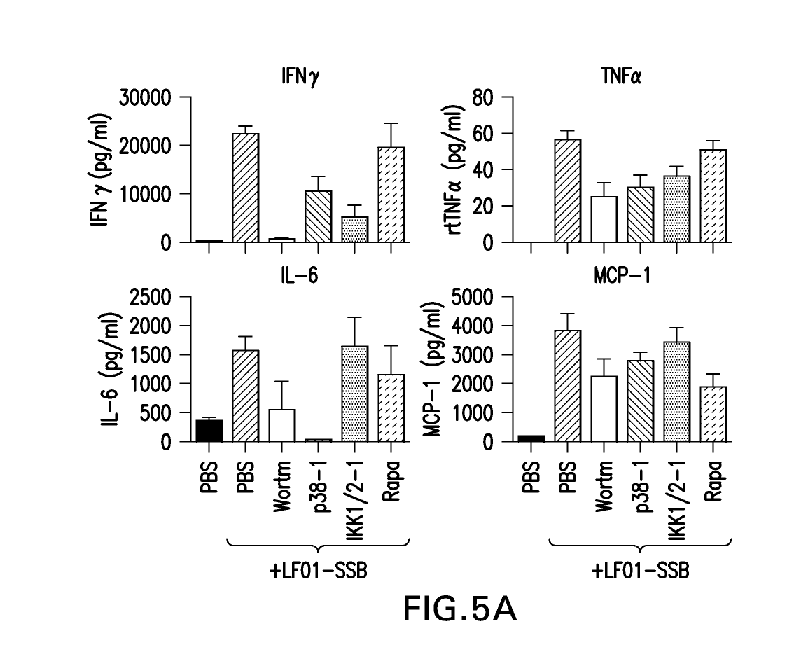

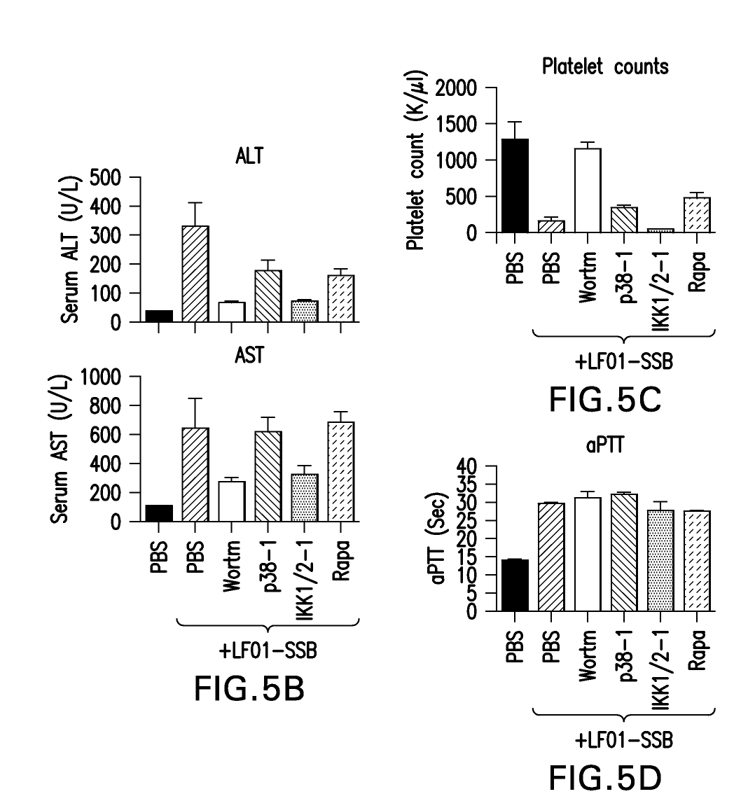

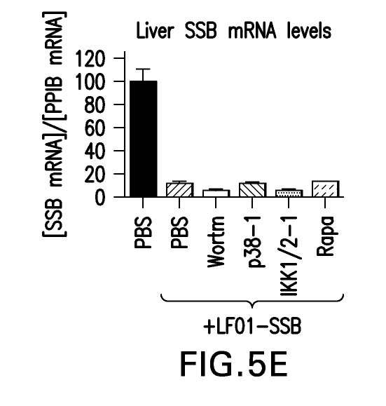

Rats (5 per group) were dosed with vehicle (PBS), Jak2-IA or dexamethasone by the regimens shown in Table 1, 1 hr prior to an IV dose of PBS or LF01-SSB (3 mg/kg). Blood and tissue samples were collected at different times post administration of LF01-SSB for various analyses as indicated in FIG. 1c. 2 out of 5 animals receiving PBS followed by LF01-SSB died by 24 hr. So, in this group samples from 3 survived animals were collected at 24 hr for analyses. No unscheduled death in other groups was detected. Bars indicate SEM.

(a) Quantification of cytokines in plasma at 3 hr post LF01-SSB treatment.

(b) Measurements of ALT and AST in serum at 24 hr.

(c) Platelet counts at 24 hr.

(d) aPTT measurements at 24 hr. See: seconds.

(e) TUNEL analysis on liver tissues. Representative images are shown. Quantification of TUNEL staining was performed using the Arial System and 9 randomly chosen fields from each animal sample were imaged and analyzed.

(f) TUNEL analysis on spleen tissues performed as described for liver tissues.

(g) Quantification of SSB mRNA relative to PpiB levels in the medial lobe of rat livers.

FIG. 4: Pretreatment with Jak2-IA abrogates LF01-ApoB-induced toxicities in rats.

Rats (5 per group) were dosed with PBS or Jak2-IA 1 hr prior to the administration of LF01-ApoB at either 3 or 9 mg/kg. Urine from all animals was collected over the course of 24 hr for visual examination of red urine, indicative of hematuria. Blood and tissue samples were collected from animals receiving 3 mg/kg LF01-ApoB for various analyses as described in FIG. 3, while animals receiving 9 mg/kg LF01-ApoB were monitored for lethality until 96 hr. The numbers of unscheduled death and animals with red urine as well as the levels of plasma cytokines at 3 hr post LF01-ApoB dose and the measurements of ALT, AST, aPTT and platelet counts at 24 hr are shown in (a). A photo of urine samples collected from animals receiving the treatments as indicated is shown in (b).

FIG. 5: The alleviative effects on LF01-SSB-induced toxicities by wortmannin, p38-I (SB-203580), IKK1/2-I (PDTC) and rapamycin in rats.

Animals (5 per group) were treated with PBS, wortmannin, p38-I, IKK1/2-1 or rapamycin 1 hr prior to the administration of PBS or FL01-SSB (3 mg/kg). Blood and tissue samples were collected for various analyses as depicted in FIG. 1c. 1 out of 5 animals receiving FL01-SSB with PBS pretreatment died by 24 hr.

(a) Quantification of cytokines in plasma at 3 hr post LF01-SSB dose.

(b) Measurements of ALT and AST in serum at 24 hr.

(c) Platelet counts at 24 hr.

(d) aPTT measurements at 24 hr. Sec: seconds.

(e) Quantification of SSB mRNA relative to PpiB levels in the medial lobe of rat livers.

### SUMMARY OF THE INVENTION

The instant invention provides a method for treating patients by administering a JAK inhibitor.

The instant invention provides a method for treating patients by administering a JAK inhibitor wherein the JAK inhibitor is a JAK2 inhibitor.

The instant invention provides a method for treating patients by administering a JAK inhibitor wherein the JAK inhibitor is selected from Jak2-IA, AG490, Pyridone 6, WP1066, LS104, TG101209, TG101348, CP690,550, CP352,664, INCB18424, WHI-P154, CMP6, SB1518, XL019, CEP-701, INCB20, AUH-6-96 and AZ960.

### DETAILED DESCRIPTION OF THE INVENTION

### Janus Kinase (JAK or “Just Another Kinase”)

Janus kinase (JAK, or “Just another kinase”) is a family of intracellular non-receptor tyrosine kinases that transduce cytokine-mediated signals via the JAK-STAT pathway. They were initially named “just another kinase” 1 & 2 (since they were just two of a large number of discoveries in a PCR-based screen of kinases), but were ultimately published as “Janus kinase”. JAKs possess two near-identical phosphate-transferring domains. One domain exhibits the kinase activity while the other negatively regulates the kinase activity of the first.

JAK1 is essential for signaling for certain type I and type II cytokines. It interacts with the common gamma chain (γe) of type I cytokine receptors, to elicit signals from the IL-2 receptor family (e.g. IL-2R, IL-7R, IL-9R and IL-15R), the IL-4 receptor family (e.g. IL-4R and IL-13R), the gp130 receptor family (e.g. IL-6R, IL-11R, LIF-R, OSM-R, cardiotrophin-1 receptor (CT-1R), ciliary neurotrophic factor receptor (CNTF-R), neurotrophin-1 receptor (NNT-1R) and Leptin-R). It is also important for transducing a signal by type I (IFN-α/β) and type II (IFN-γ) interferons, and members of the IL-10 family via type H cytokine receptors. Jak1 plays a critical role in initiating responses to multiple major cytokine receptor families. Loss of Jak1 is lethal in neonatal mice, possibly due to difficulties suckling.

Janus kinase 2 (commonly called JAK2) has been implicated in signaling by members of the type II cytokine receptor family (e.g. interferon receptors), the GM-CSF receptor family (IL-3R, IL-5R and GM-CSF-R), the gp130 receptor family (e.g. IL-6R), and the single chain receptors (e.g. Epo-R, Tpo-R, GH-R, PRL-R). JAK2 signaling is activated downstream from the prolactin receptor.

JAK3 functions in signal transduction and interacts with members of the STAT (signal transduction and activators of transcription) family. JAK3 is predominantly expressed in immune cells and transduces a signal in response to its activation via tyrosine phosphorylation by interleukin receptors. Mutations that abrogate Janus kinase 3 function cause an autosomal SCID (severe combined immunodeficiency disease). Since JAK3 expression is restricted mostly to hematopoietic cells, its role in cytokine signaling is thought to be more restricted than other JAKs. It is most commonly expressed in T cells and NK cells, but has been induced in other leukocytes, including monocytes. Jak3 is involved in signal transduction by receptors that employ the common gamma chain (γC) of the type I cytokine receptor family (e.g. IL-2R, IL-4R, IL-7R, IL-9R, IL-15R, and IL-21R). Mutations of JAK3 result in severe combined immunodeficiency (SCID). Mice that do not express JAK3 have T-cells and B-cells that fail to respond to many cytokines.

The instant invention provides a method for treating a patient, wherein the patient will be or is currently being treated with a lipid-based nucleic acid therapeutic, by administering a JAK inhibitor.

The instant invention further provides a method as described above wherein the JAK inhibitor is a JAK2 inhibitor.

The instant invention further provides a method as described above wherein the JAK inhibitor is selected from Jak2-IA, AG490, Pyridone 6, WP1066, LS104, TG101209, TG101348, CP690,550, CP352,664, INCB18424, WHI-P154, CMP6, SB1518, XL019, CEP-701, INCB20, AUH-6-96 and AZ960.

The instant invention further provides a method as described above wherein the patient is pre-treated with a JAK inhibitor.

The instant invention further provides a method as described above wherein the patient is co-treated with a JAK inhibitor.

### DEFINITIONS

The term “patient(s)” means a mammal in need of disease treatment wherein the mammal is administered a lipid-based nucleic acid therapeutic for that disease. In particular, the term “patient(s)” means a human in need of disease treatment wherein the human is administered a lipid-based nucleic acid therapeutic for that disease. For clarity, a “patient” means a mammal that is currently or will be treated with a lipid-based nucleic acid therapeutic.

It is understood that a patient may be 1) pre-treated (administration of a JAK inhibitor prior to the administration of the lipid-based nucleic acid therapeutic); 2) co-treated (administration of a JAK inhibitor at the same time as the administration of the lipid-based nucleic acid therapeutic); or 3) a combination thereof. It is understood that a patient may be administered a JAK inhibitor prior to onset of treatment with a lipid-based nucleic acid therapeutic or following treatment with lipid-based nucleic acid therapeutic. In addition, a JAK inhibitor may be administered during the period of administration of a lipid-based nucleic acid therapeutic but does not need to occur over the entire treatment period of a lipid-based nucleic acid therapeutic.

The term “mammal”, in particular, means a human.

The term “lipid-based” means liposomes (including LNPs and SNALPs), lipoplexes, and any drug, RNA or gene delivery vehicles, microparticles, and nanoparticles containing a lipid component comprising cationic lipids, neutral lipids, anionic lipids, biodegradable lipids or PEG lipids. In an embodiment, “lipid-based” means liposomes.

The term “nucleic acid” means oligonucleotides, enzymatic nucleic acids, antisense nucleic acids, triplex forming oligonucleotides, 2,5-A chimeras, allozymes, aptamers, decoys and analogs thereof, and small nucleic acid molecules, such as short interfering nucleic acid (siNA), short interfering RNA (siRNA), double-stranded RNA (dsRNA), micro-RNA (miRNA), and short hairpin RNA (shRNA) molecules. In an embodiment, “nucleic acid” means an miRNA or a siRNA. In another embodiment, “nucleic acid” means a siRNA. It is understood that a “lipid-based nucleic acid therapeutic” may contain combinations of the above described “nucleic acids”. For example, a “lipid-based nucleic acid therapeutic” may contain both miRNA and siRNA.

The term “JAK inhibitor” means any small molecule compound, antibody, siRNA or vaccine that inhibits JAK (including JAK1, JAK2, JAK3 and TYK2). In an embodiment, “JAK inhibitor” means any small molecule compound, antibody, siRNA or vaccine that inhibits JAK (including JAK1, JAK2 and JAK3). In another embodiment, “JAK inhibitor” means any small molecule compound, antibody, siRNA or vaccine that inhibits JAK I. In another embodiment, “JAK inhibitor” means any small molecule compound, antibody, siRNA or vaccine that inhibits JAK2. In another embodiment, “JAK inhibitor” means any small molecule compound, antibody, siRNA or vaccine that inhibits JAK3. In another embodiment, “JAK inhibitor” means any small molecule compound, antibody, siRNA or vaccine that inhibits JAK1/2.

In a further embodiment, the term “JAK inhibitor” means any small molecule compound that inhibits JAK (including JAK1, JAK2, JAK3 and TYK2). In an embodiment, “JAK inhibitor” means any small molecule compound that inhibits JAK (including JAK1, JAK2 and JAK3). In another embodiment, “JAK inhibitor” means any small molecule compound that inhibits JAK1. In another embodiment, “JAK inhibitor” means any small molecule compound that inhibits JAK2. In another embodiment, “JAK inhibitor” means any small molecule compound that inhibits JAK3. In another embodiment, “JAK inhibitor” means any small molecule compound that inhibits JAK1/2.

JAK inhibitors include phenylaminopyrimidine compounds (WO2009/029998), substituted tricyclic heteroaryl compounds (WO2008/079965), cyclopentyl-propanenitrile compounds (WO2008/157208 and WO2008/157207), indazole derivative compounds (WO2008/114812), substituted amino-thiophene carboxylic acid amide compounds (WO2008/156726), naphthyridine derivative compounds (WO2008/112217), quinoxaline derivative compounds (WO2008/148867), pyrrolopyrimidine derivative compounds (WO2008/119792), purinone and imidazopyridinone derivative compounds (WO2008/060301), 2,4-pyrimidinediamine derivative compounds (WO2008/118823), deazapurine compounds (WO2007/117494) and tricyclic heteroaryl compounds (WO2008/079521).

JAK inhibitors include compounds disclosed in the following publications: US2004/176601, US2004/038992, US2007/135466, US2004/102455, WO2009/054941, US2007/134259, US2004/265963, US2008/194603, US2007/207995, US2008/260754, US2006/063756, US2008/261973, US2007/142402, US2005/159385, US2006/293361, US2004/205835, WO2008/148867, US2008/207613, US2008/279867, US2004/09799, US2002/055514, US2003/236244, US2004/097504, US2004/147507, US2004/176271, US2006/217379, US2008/092199, US2007/043063, US2008/021013, US2004/152625, WO2008/079521, US2009/186815, US2007/203142, WO2008/144011, US2006/270694 and US2001/044442.

JAK inhibitors further include compounds disclosed in the following publications: WO2003/011285, WO2007/145957, WO2008/156726, WO2009/035575, WO2009/054941, and WO2009/075830. JAK inhibitors further include compounds disclosed in the following patent applications: U.S. Ser. Nos. 61/137,475 and 61/134,338.

A JAK inhibitor further includes Pyridone 6 as described in Bioorganic. Med. Chem. Letters (2002) 12:1219-1223.

Specific JAK inhibitors include Jak2-IA, AG490, Pyridone 6, WP1066, LS104, TG101209, TG101348, CP690,550, CP352,664, INCB18424, WHI-P154, CMP6, SB151S, XL019, CEP-701, INCB20, AUH-6-96 and AZ960.

Specific JAK inhibitors are Jak2-IA and CP690,550.

### Lipid-Based

Since the first description of liposomes in 1965, by Bangham (J. Mal. Biol. 13, 238-252), there has been a sustained interest and effort in the area of developing lipid-based carrier systems for the delivery of pharmaceutically active compounds. Liposomes are attractive drug carriers since they protect biological molecules from degradation while improving their cellular uptake. One of the most commonly used classes of liposome formulations for delivering polyanions (e.g., DNA) is that which contains cationic lipids. Lipid aggregates can be formed with macromolecules using cationic lipids alone or including other lipids and amphiphiles such as phosphatidylethanolamine. It is well known in the art that both the composition of the lipid formulation as well as its method of preparation have effect on the structure and size of the resultant anionic macromolecule-cationic lipid aggregate. These factors can be modulated to optimize delivery of polyanions to specific cell types in vitro and in vivo. The use of cationic lipids for cellular delivery of biologically active molecules has several advantages. The encapsulation of anionic compounds using cationic lipids is essentially quantitative due to electrostatic interaction. In addition, it is believed that the cationic lipids interact with the negatively charged cell membranes initiating cellular membrane transport (Akhtar et al., 1992, Trends Cell Bio., 2, 139; Xu et al., 1996, Biochemistry 35, 5616).

Various lipid nucleic acid particles and methods of preparation thereof are described in U.S. Patent Application Publication Nos. 2003/0077829, 2003/0108886, 2006/0051405, 2006/0083780, 2003/0104044, 2006/0051405, 2004/0142025, 2006/00837880, 2005/0064595, 2005/0175682, 2005/0118253, 2005/0255153 and 2005/0008689; and U.S. Pat. Nos. 5,885,613; 6,586,001; 6,858,225; 6,858,224; 6,815,432; 6,586,410; 6,534,484; and 6,287,591.

Vagle et al., U.S. Patent Application Publication No. 2006/0240554 describes lipid nanoparticle based compositions and methods for the delivery of nucleic acids.

“Lipid-based” further comprises lipid nanoparticles or LNP compositions, see for example LNP compositions described in U.S. Patent Application Publication No. 2006/0240554.

“Lipid-based” further comprises stable nucleic acid particles or SNALP compositions, see for example International PCT Publication No. WO2007/012191, and U.S. Patent Application Publication Nos. 2006/083780, 2006/051405, US2005/175682, US2004/142025, US2003/077829 and US2006/240093.

“Lipid-based” further comprises delivery systems as described in International PCT Publication Nos. WO2005/105152 and WO2007/014391, and U.S. Pat. Nos. 7,148,205, 7,144,869, 7,138,382, 7,101,995, 7,098,032, 7,098,030, 7,094,605, 7,091,041, 7,087,770, 7,071,163, 7,049,144, 7,049,142, 7,045,356, 7,033,607, 7,022,525, 7,019,113, 7,015,040, 6,936,729, 6,919,091, 6,897,068, 6,881,576, 6,872,519, 6,867,196, 6,818,626, 6,794,189, 6,740,643, 6,740,336, 6,706,922, 6,673,612, 6,630,351, 6,627,616, 6,593,465, 6,458,382, 6,429,200, 6,383,811, 6,379,966, 6,339,067, 6,265,387, 6,262,252, 6,180,784, 6,126,964, 6,093,701, and 5,744,335.

“Lipid-based” further comprises peptide or peptide related delivery systems, see for example U.S. Patent Application Publication Nos. 2006/0040882, 2005/0136437, 2005/0031549, and 2006/0062758.

“Lipid-based” further comprises albumin, collagen, and gelatin, polysaccharides such as dextrans and starches, and matrix forming compositions including polylactide (PLA), polyglycolide (PGA), lactide-glycolide copolymers (PLG), poly(lactic-co-glycolic acid) (PLGA), polycaprolactone, lactide-caprolactone copolymers, polyhydroxybutyrate, polyalkylcyanoacrylates, polyanhydrides, polyorthoesters, acrylate polymers and copolymers such as methyl methacrylate, methacrylic acid, hydroxyalkyl acrylates and methacrylates, ethylene glycol dimethacrylate, acrylamide and/or bisacrylamide, cellulose-based polymers, ethylene glycol polymers and copolymers, oxyethylene and oxypropylene polymers, poly(vinyl alcohol), polyvinylacetate, polyvinylpyrrolidone, polyvinylpyridine, and/or any combination thereof.

The instant invention provides a method for treating patients by administering a JAK inhibitor.

The instant invention provides a method for treating patients by administering a JAK inhibitor wherein the JAK inhibitor is a JAK2 inhibitor.

The instant invention provides a method for treating patients by administering a JAK inhibitor wherein the JAK inhibitor is selected from Jak2-IA, AG490, Pyridone 6, WP1066, LS104, TG101209, TG101348, CP690,550, CP352,664, INCB18424, WHI-P154, CMP6, SB1518, XL019, CEP-701, INCB20, AUH-6-96 and AZ960.

### General Oligonucleotide Synthesis

Oligonucleotides may be conveniently and routinely made through the well-known technique of solid phase synthesis. Equipment for such synthesis on scales from small to large is sold by several vendors including, for example, Applied Biosystems (Foster City, Calif.), GE Healthcare (US and UK). Any other means for such synthesis known in the art may additionally or alternatively be employed. It is well known to use similar techniques to prepare oligonucleotides on all scales. Further, synthesis of oligonucleotides is described in the following references (Ohkubo et al., 2006 Curr. Protoc. Nucleic Acid Chem., Chapter 3:Unit 3.15; PCT WO 1996/040708; U.S. Pat. Nos. 4,458,066 and 4,973,679; Beaucage et al., 1992 Tetrahedron Lett. 22:1859-69; U.S. Pat. No. 4,415,732).

### General RNA Synthesis

Methods of RNA synthesis are well known in the art (Scaringe, S. A. Ph.D. Thesis, University of Colorado, 1996; Scaringe, S. A., et al., J. Am. Chem. Soc., 1998, 120, 11820-11821; Matteucci, M. D. and Caruthers, M. H. J. Am. Chem. Soc., 1981, 103, 3185-3191; Beaucage, S. L. and Caruthers, M. H. Tetrahedron Lett., 1981, 22, 1859-1862; Dahl, 13. J., et al., Acta Chem. Scand., 1990, 44, 639-641; Reddy, M. P., et al., Tetrahedron Lett., 1994, 25, 4311-4314; Wincott, F. et al., Nucleic Acids Res., 1995, 23, 2677-2684; Griffin, 13. E., et al., Tetrahedron, 1967, 23, 2301-2313; Griffin, B. E., et al., Tetrahedron, 1967, 23, 2315-2331).

Once synthesized, complementary RNA oligonucleotides can then be annealed by methods known in the art to form double stranded (duplexed) oligonucleotide compounds. For example, duplexes can be formed by combining 30 μl of each of the complementary strands of RNA oligonucleotides (50 μM RNA oligonucleotide solution) and 15 μl of 5× annealing buffer (100 mM potassium acetate, 30 mM HEPES-KOH pH 7.4, 2 mM magnesium acetate) followed by heating for 1 minute at 90° C., then 1 hour at 37° C. The resulting duplexed RNA oligonucleotides can be used in kits, assays, screens, or other methods to investigate the role of a target nucleic acid, or for diagnostic or therapeutic purposes.

### General siRNA Synthesis

RNA oligonucleotides can be synthesized in a stepwise fashion comprising at least one nucleosidic phosphoramidate linkage derived from nucleosidic phosphoramidites. Each nucleotide can be added sequentially (3′- to 5′-direction) to a solid support-bound oligonucleotide. The first nucleoside at the 3′-end of the chain can be covalently attached to a solid support. The 5′-O-dimethoxy trityl group of the nucleoside bound to the solid support is removed by treatment with an acid such dichloroacetic acid. The nucleotide precursor, a nucleosidic phosphoramidite, and activator can be added, coupling the second base onto the 5′-end of the first nucleoside. The linkage may be then oxidized to the more stable and ultimately desired P(V) linkage. The support is washed and any unreacted 5′-hydroxyl groups can be capped with acetic anhydride to yield 5′-acetyl moieties. The cycle can be repeated for each subsequent nucleotide. This cycle is repeated until the desired oligonulcoetide sequence has been completed.

Following synthesis, the support bound oligonucleotide can be treated with a base such a diethylamine to remove the cyanoethyl protecting groups of the phosphate backbone. The support may then be treated with a base such as aqueous methylamine. This releases the oligonucleotides into solution, deprotects the exocyclic amines. Any 2′ silyl protecting groups can be removed by treatment with fluoride ion. The oligonucleotide can be analyzed by anion exchange HPLC at this stage.

The oligonucleotides synthesized by this method can be purified by HPLC. Once purified complementary RNA oligonucleotides can then be annealed by methods known in the art to form double stranded (duplexed) oligonucleotide compounds.

### Specific siRNA Synthesis

### Solid Phase Synthesis

The single-strand oligonucleotides are synthesized using phosphoramidite chemistry on an automated solid-phase synthesizer. An adjustable synthesis column is packed with solid support derivatized with the first nucleoside residue. Synthesis is initiated by detritylation of the acid labile 5′-O-dimethoxytrityl group to release the 5′-hydroxyl. Phosphoramidite and a suitable activator (in acetonitrile) are delivered simultaneously to the synthesis column resulting in coupling of the amidite to the 5′-hydroxyl (the column is then washed with acetonitrile). Oxidizers such as I₂ are pumped through the column to oxidize the phosphite triester linkage P(III) to its phosphotriester P(V) analog. Alternately, sulfurizing reagent (in acetonitrile) replaces the iodine solution when a phosphorothioate triester linkage is required by the sequence. Unreacted 5′-hydroxyl groups are capped using reagents such as acetic anhydride in the presence of 2,6-lutidine and N-methylimidazole. The elongation cycle resumes with the detritylation step for the next phosphoramidite incorporation. This process is repeated until the desired sequence has been synthesized. The synthesis concludes with the removal of the terminal dimethoxytrityl group.

### Cleavage and Deprotection

On completion of the synthesis, the solid support and associated oligonucleotide is filtered, dried under vacuum and transferred to a reaction vessel. Aqueous base is added and the mixture is heated to effect cleavage of the succinyl linkage, removal of the cyanoethyl phosphate protecting group and the exocyclic amine protecting groups. The mixture is filtered under vacuum to remove the solid support. The solid support is rinsed with DMSO which is combined with the filtrate. The mixture is cooled, fluoride reagent such as triethylamine trihydrofluoride is added and the solution is heated. The reaction is quenched with suitable buffer to provide a solution of crude single strand product.

### Anion Exchange Purification

The oligonucleotide strand is purified using chromatographic purification. The product is eluted using a suitable buffer gradient. Fractions are collected in closed sanitized containers, analyzed by HPLC and the appropriate fractions are combined to provide a pool of product which is analyzed for purity (HPLC), identity (HPLC and LCMS) and concentration (UV A₂₆₀).

### Annealing

Based on the analysis of the pools of product, equal molar amounts (calculated using the theoretical extinction coefficient) of the sense and antisense oligonucleotide strands are transferred to a reaction vessel. The solution is mixed and analyzed for purity of duplex by chromatographic methods. If the analysis indicates an excess of either strand, then additional non-excess strand is titrated until duplexing is complete. When analysis indicates that the target product purity has been achieved, the material is transferred to the Tangential Flow Filtration (TFF) system for concentration and desalting.

### Ultrafiltration

The annealed product solution is concentrated using a TFF system containing an appropriate molecular weight cut-off membrane. Following concentration, the product solution is desalted via diafiltration using WFI quality water until the conductivity of the filtrate is that of water.

### Lyophilization

The concentrated solution is transferred to sanitized trays or containers in a shelf lyophilizer. The product is then freeze-dried to a powder. The trays are removed from the lyophilizer.

### Formulations

### JAK Inhibitors

Pharmaceutical compositions of JAK inhibitors may be administered in a number of ways depending upon whether local or systemic treatment is desired and upon the area to be treated. Administration may be topical (including but not limited to ophthalmic and to mucous membranes including vaginal and rectal delivery), pulmonary, e.g., by inhalation or insufflation of powders or aerosols, including by nebulizer (intratracheal, intranasal, epidermal and transdermal), oral or parenteral. Formulations for JAK inhibitors may be in a form suitable for oral or parenteral use. Compositions intended for oral or parenteral use may be prepared according to any method known to the art for the manufacture of pharmaceutical compositions. The pharmaceutical compositions may be in the form of sterile injectable aqueous solutions.

One of skill in the art will recognize that formulations are routinely designed according to their intended use, i.e. route of administration.

### Nucleic Acids

Formulations for oligonucleotides and siRNA are well known in the art (U.S. Pat. Nos. 6,559,129, 6,042,846, 5,855,911, 5,976,567, 6,815,432, and 6,858,225 and US 2006/0240554, US 2008/0020058 and PCT/US08/002,006).

Pharmaceutical compositions of oligonucleotides may be administered in a number of ways depending upon whether local or systemic treatment is desired and upon the area to be treated. Administration may be topical (including but not limited to ophthalmic and to mucous membranes including vaginal and rectal delivery), pulmonary, e.g., by inhalation or insufflation of powders or aerosols, including by nebulizer (intratracheal, intranasal, epidermal and transdermal), oral or parenteral.

Parenteral administration includes intravenous, intraarterial, subcutaneous, intraperitoneal or intramuscular injection or infusion; or intracranial, e.g., intrathecal or intraventricular, administration. Sites of administration are known to those skilled in the art.

One of skill in the art will recognize that formulations are routinely designed according to their intended use, i.e. route of administration.

### Dosing

The formulation of therapeutic compositions and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates.

### JAK Inhibitors

Optimum dosages may vary depending on the relative potency of individual JAK inhibitors, and can generally be estimated based on EC₅₀s found to be effective in in vitro and in vivo animal models. In an embodiment, a suitable amount of an inhibitor of JAK is administered to a mammal undergoing treatment for cancer. Administration occurs in an amount of inhibitor of between about 0.1 mg/kg of body weight to about 60 mg/kg of body weight per day, or between 0.5 mg/kg of body weight to about 40 mg/kg of body weight per day. Another therapeutic dosage that comprises the instant composition includes from about 0.01 mg to about 1000 mg of inhibitor of JAK. In another embodiment, the dosage comprises from about 1 mg to about 1000 mg of inhibitor of JAK.

### Nucleic Acids

Optimum dosages may vary depending on the relative potency of individual oligonucleotides, type of lipid-based delivery vehicle, species differences, etc., and can generally be estimated based on EC₅₀s found to be effective in in vitro and in vivo animal models. In general, dosage is from 0.01 ug to 100 g per kg of body weight, from 0.1 μg to 10 g per kg of body weight, from 1.0 μg to 1 g per kg of body weight, from 10.0 μg to 100 mg per kg of body weight, from 100 μg to 10 mg per kg of body weight, or from 1 mg to 5 mg per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 ug to 100 g per kg of body weight, once or more daily, to once every 20 years.

### Indications

### JAK Inhibitors

It is well known in the art that JAK inhibitors are useful therapeutically in mammals, in particular humans. JAK inhibitors are useful for treating hypertension, ischaemia, allergic asthma, multiple sclerosis, glomerulonephritis, allograft rejection, graft versus host disease, autoimmune diseases, RA, polycythemia vera, essential thrombocythemia, sarcoma, other myeloproliferative disorders, leukaemia, lymphoma, cardiac and neurodegenerative disorders and COPD.

### Nucleic Acids

It is well known in the art that oligonucleotides (including antisense, siRNA and miRNA) are useful therapeutically in mammals, in particular humans (Karagiannis, T. and El-Osta, A., 2004 Cancer Biol. Ther., 3:1069-74; Karagiannis, T. and El-Osta, A., 2005 Cancer Gene Ther., 12:787-95; Dallas, A. and Vlassov A., 2006 Med. Sci. Monit., 12:67-74; Spurgers et al., 2008 Antiviral Research, 78:26-36; Fuchs et al., 2004 Curr. Mol. Med., 4:507-17; Eckstein, F., 2007 Expert Opin. Biol. Ther., 7:1021-34).

While the present invention has been described with specificity in accordance with certain of its preferred embodiments, the following examples serve only to illustrate the invention and are not intended to limit the same. Each of the references, GenBank accession numbers, and the like recited in the present application is incorporated herein by reference in its entirety.

### Examples

### Introduction

A new liposomal formulation (LF01) has been developed for liver delivery of siRNA via systemic administration. While LF01-formulated siRNA nanoparticles exhibited robust efficacy in silencing several liver targets in animals, including apolipoprotein B (ApoB) and La antigen (SSB), a ubiquitously expressed gene involved in the maturation of tRNA precursors³¹, they triggered multi-systemic toxicities and lethality, leaving a narrow therapeutic window. This is despite the fact that all siRNA payloads are sequence-selected and chemically modified to attenuate the immunostimulatory activity. Using LF01-encapsulated SSB siRNA (LF01-SSB) or ApoB siRNA (LF01-ApoB), we investigated the etiology of LF01-siRNA-triggered pathological responses by determining the activity of three classes of anti-inflammation reagents in mitigating LF01-siRNA-induced lethality and toxicities in rats: 1) antagonists of Jak2, p38, IKK1/2, PI3K and mTOR that block different pathways of the innate immune response, 2) dexamethasone, a multifunctional suppressor of inflammation^{32, 33}, and FK506, an immunosuppressant inhibiting the activation of nuclear factor of activated T cells (NFAT)³⁴ and 3) inhibitors of two effectors of inflammation, cyclooxygenase-2 (COX2)³⁵ and inducible nitric oxide synthase (iNOS)³⁶. We demonstrated that activation of innate immunity is a primary trigger of multi-systemic toxicities. While the inhibition of an individual pathway linked to TLRs is not sufficient to eliminate cytokine induction and subsequent toxic responses, Jak2 inhibitors can block the production and function of a group of cytokines and abrogate liposomal siRNA-associated toxicities.

### Results

LF01-siRNA Nanoparticles are Efficacious but Toxic in Rodents

LF01 consists of a cationic lipid, cholesterol-linolyl dimethyl amine (CLinDMA), cholesterol and dimethylglycerol-polyethylene glycol (DMG-PEG) lipid at a molar ratio of 60:38:2 (FIG. 1a). When assembled with either SSB or ApoB siRNA, the mean nanoparticle size is ˜170 nm in diameter with +10 mV surface charge, and the siRNA encapsulation efficiency is >90% with total lipid:siRNA ratio=12:1 (wt:wt). Both SSB and ApoB siRNAs are chemically modified as previously described to increase nuclease resistance and reduce immunostimulatory activity³⁰. All liposomal-siRNA preparations were examined for potential endotoxin contamination using a FDA-approved method to ensure that the endotoxin levels, if any, were below the endotoxin release limit defined for humans by FDA and WHO as described in Methods. Both LF01-SSB and LF01-ApoB are potent in silencing target gene expression, with IC₅₀ values of 0.52 nM and 0.76 nM toward SSB and ApoB respectively in cultured HepG2 cells after 24 hr treatment. In rodents, a single intravenous (IV) dose of LF01-SSB or LF01-ApoB at 1 mg/kg (siRNA dose) caused >70% reduction in liver SSB or ApoB mRNA levels specifically (FIG. 1b). To characterize LF01-siRNA-linked toxicities, rats were IV dosed with 3 or 9 mg/kg of LF01-SSB or PBS as control and then monitored for adverse responses as illustrated in FIG. 1c. Multifaceted toxicities were detected, including 1) lethality (all animals dosed with 9 mg/kg of LF01-SSB died by 24 hr post dosing), 2) induction of cytokines in plasma 3 hr post dosing, including IL6, TNF-γ, TNF-α and MCP-1, 3) elevation of serum alanine aminotransferase (ALT) and aspartate aminotransferase (AST), 4) thrombocytopenia, 5) coagulopathy, manifested by the elongation of activated partial thromboplastin time (aPTT) and 6) red urine, indicative of severe hematuria (FIG. 1d). No changes in erythrocyte counts, hemoglobin and hematocrit measurements and prothrombin time (PT) were detected (data not shown). Since there is no sign of significant hemolysis, the appearance of bloody urine indicates the impairment of renal function. LF01-ApoB caused comparable toxic responses (Supplementary Table 1), indicating that the detected toxicities are independent of siRNA sequences or target gene repression. Finally, the majority of such toxicities were also observed in mice (data not shown).

Identification of Jak2 Inhibitors and Dexamethasone as Suppressors of LF01-siRNA-Induced Lethality in Rats

Since LF01-siRNA nanoparticles provoked lethality and multifactorial toxicities involving different systems in rats, this offers an in vivo model for pharmacologically probing the mechanism that triggers LF01-siRNA toxicities. We first evaluated the suppression of LF01-SSB-induced lethality and visible hematuria by anti-inflammation drugs and pathway-specific inhibitors of the innate immune system as shown in Table 1. The in vivo pharmacological activities of these reagents were reported except for Jak2-IA, a potent and selective Jak2 inhibitor developed by Merck. The structure, pharmacodynamic and pharmacokinetic activities of Jak2-IA in rodents are shown in FIG. 2. Co-treatment with Jak2-IA at 60 mg/kg, p.o. caused >8 hr profound suppression of Jak2-mediated phosphorylation of STAT5 in blood cells following stimulation by Aranesp, a homolog of erythropoietin (FIG. 2b). The target inhibitory activities of Jak2-IA and other reagents and their efficacious dosing regimens identified from former studies, that caused robust pharmacodynamic (PD) effect in rodents, are shown in Table 1^{34, 35, 37-46}. Rats were pre-dosed with either a vehicle or one of these reagents using the dosing regimen listed in Table 1, followed by an IV dose of LF01-SSB at 9 mpk 1 hr later, and animals were monitored for lethality for 96 hr. In addition, urine was collected over the course of 24 hr for visual observation of hematuria. As shown in Table 1, Jak2-IA and dexamethasone completely prevented LF01-SSB-induced lethality and visible hematuria whereas inhibitors of PI3K, p38, IKK1/2 and mTOR exhibited partial rescue effect by reducing mortality and/or the occurrence of hematuria. On contrary, FK506, COX2 or iNOS antagonist showed no protective activity. To preclude the possibility that Jak2-IA-mediated rescue is linked to an unknown property of this compound rather than Jak2 inhibition, we tested another Jak2/3 dual inhibitor, CP-690550⁴⁵, and found that CP-690550 also prevented LF01-SSB-induced lethality and visible hematuria (Table 1), confirming that Jak2 inhibition suppresses LF01-SSB lethality.

Jak2 Inhibitors and Dexamethasone Abrogated Cytokine Release and Multi-Systemic Toxicities Induced by LF01-siRNA Nanoparticles

As Jak2 plays a central role in mediating functions of a group of cytokines^{25, 47}, identification of Jak2 inhibitors as a suppressor of LF01-siRNA lethality suggests that Jak2-mediated cytokine response is either a trigger or an essential executor of LF01-siRNA-associated toxicities. In the former scenario, Jak2 inhibitors should be able to alleviate not only cytokine response but also other toxicities. To test if this is the case, rats were treated with PBS, Jak2-IA or dexamethasone 1 hr prior to intravenous administration of LF01-SSB at 3 mg/kg. Blood was collected by retro-orbital bleed 3 hr post injection of LF01-SSB for cytokine assessment and animals were sacrificed at 24 hr for collection of blood and tissues for various analyses as depicted in FIG. 1c. Among rats treated with PBS followed by LF01-SSB (n=5), 2 animals died by 24 hr and the survived animals displayed multiple abnormalities (FIG. 3), recapitulating previous observations (FIG. 1d). Pre-treatment with either Jak2-IA or dexamethasone not only prevented lethality (no death), but also suppressed all toxic responses including cytokine induction, ALT and AST elevation, thrombocytopenia, elongation of aPTT as well as hepatic and splenic cell death as assessed by TUNEL analysis (FIG. 3a-f). Importantly, pre-treatment with either Jak2-IA or dexamethasone did not affect LF01-SSB mediated SSB gene silencing (FIG. 3g), disconnecting LF01-SSB efficacy from its toxicities. As LF01-ApoB caused similar toxicities as LF01-SSB (Supplementary Table 1), we also evaluated the mitigation of LF01-ApoB toxicities by Jak2-IA and found that Jak2-IA showed similar protection against LF01-ApoB toxicities (FIG. 4), suggesting that Jak2-IA mediated protection is independent of siRNA sequences encapsulated in liposomes. Moreover, we evaluated the activity of CP-690550 in mitigating LF01-SSB toxicities and observed the suppression of toxic responses by this Jak2/3 inhibitor (Supplementary Table 2). This observation is consistent with its ability in preventing LF01-SSB lethality (Table 1). Treatment with Jak2-IA or CP-690550 alone caused no adverse effects (data not shown).

Inhibitors of PI3K, mTOR, p38 and IKK1/2 Partially Mitigated LF01-siRNA-Induced toxicities

Inhibitors of PI3K, mTOR, p38 and IKK1/2 exhibited partial alleviation on LF01-SSB-induced lethality and visible hematuria (Table 1). It is interesting to examine their abilities in mitigating other pathologies triggered by LF01-SSB. As described above, rats were pre-dosed with PBS or one of these inhibitors 1 hr prior to an IV dose of LF01-SSB at 3 mg/kg, and animals were monitored for various toxic responses. Whereas pre-treatment with one of these inhibitors attenuated cytokine induction and/or ALT/AST elevation to different extents, only wortmannin prevented thrombocytopenia (FIG. 5c). Unlike Jak2-IA, wortmannin, rapamycin, p38-I or IKK1/2-I showed no mitigation effect on coagulopathy (elongation of aPTT) (FIG. 5d). Finally, none of these inhibitors interfered with LF01-SSB-induced SSB gene silencing (FIG. 5e).

### FK506, Etoricoxib and Aminoguanidine Displayed No Alleviation on LF01-SSB Nanoparticle-Mediated Toxicities

Although FK506, etoricoxib and aminoguanidine were inactive in suppressing LF01-SSB lethality, we further evaluated their activities in mitigating LF01-SSB-triggered toxic responses. One hour after a pretreatment with one of these agents, rats were dosed with LF01-SSB at 3 mg/kg and the toxicities were monitored as described above (FIG. 1c). As summarized in Supplementary Table 3, these agents caused no appreciable suppression of cytokine release, elevation of serum transaminases, thrombocytopenia or coagulopathy induced by LF01-SSB. There results are consistent with the incompetence of these agents in mitigating LF01-SSB lethality (Table 1).

TABLE 1
Suppression of LF01-SSB-induced lethality and visible hematuria
by different inhibitors.

| | | | Rescue effect + | |
|---|---|---|---|---|
| | | | inhibitor/− | |
| | | Dosing | inhibitor | |
| Inhibitors | Biological activity | regimen | Death | Red urine |
| Dexamethasone | Pan-inhibitor of | 6 mg/kg, | 0/5 | 0/5 |
| | immune response | i.p. | (n = 5) | (n = 5) |
| Jak2-IA | Jak2 inhibitor | 60 mg/kg | 0/5 | 0/5 |
| | Jak2 IC₅₀ = 1 nM | p.o. | (n = 5) | (n = 5) |
| | Jak3 IC₅₀ = 220 | | | |
| | nM | | | |
| CP-690550 | Jak2/3 dual | 15 mg/kg | 0/4 | 0/4 |
| | inhibitor | s.c. BID, q6h | (n = 5) | (n = 5) |
| | Jak2 IC₅₀ = 20 nM | | | |
| | Jak3 IC₅₀ = 1 nM | | | |
| Wortmaninn | Pan-inhibitor of | 1.5 mg/kg | 2/4 | 1/4 |
| | PI3K | i.p. | (n = 5) | (n = 5) |
| | PI3K IC₅₀ < 5 nM | | | |
| SB-203580 | Inhibitor of p38 | 100 mg/kg | 2/5 | 2/5 |
| (p38-I) | p38α IC₅₀ = 48 nM | p.o. | n = 5 | n = 5 |
| PDTC | Pan-inhibitor of | 100 mg/kg | 4/5 | 3/5 |
| (IKK2-1) | IKK, IKK1 IC₅₀ = | i.p. | (n = 5) | (n = 5) |
| | 400 nM, IKK2 | | | |
| | IC₅₀ = 20 nM | | | |
| Rapamycin | Inhibitor of | 10 mg/kg | 4/5 | 4/5 |
| | mTOR | p.o. | (n = 5) | (n = 5) |
| FK506 | Inhibitor of NFAT | 5 mg/kg | 5/4 | 4/4 |
| | | i.p. | (n = 5) | (n = 5) |
| Etoricoxib | COX2 inhibitor | 20 mg/kg | 4/3 | 4/4 |
| (COX2-I) | COX2 IC₅₀ = nM | p.o. | (n = 5) | (n = 5) |
| Aminoguanidine | Inhibitor of iNOS | 400 mg/kg | 4/3 | 2/3 |
| (AG) | | i.p. | (n = 4) | (n = 4) |

Rats were dosed with PBS or one of these inhibitors 1 hr prior to IV administration of PBS or FL01-SSB at 9 mg/kg. Urine samples were collected over a course of 24 hr for visual examination of hematuria (red urine). Animals were monitored for lethality until 96 hr post LF01-SSB dose. Unscheduled deaths and cases of visible hematuria in animals receiving LF01-SSB with or without inhibitor pretreatment were scored and presented.

SUPPLEMENTARY TABLE 1
Systemic administration of LF01-ApoB causes lethality and multifaceted toxicities in rats.

| | | Red | AST | ALT | Platelets | apTT | Cytokines (pg/ml) | | | |
|---|---|---|---|---|---|---|---|---|---|---|
| | Death | urine | (U/L) | (U/L) | (K/μl) | (Sec) | IFNγ | IL-6 | MCP-1 | TNFα |
| PBS | 0 | 0 | 118 ± 9 | 28 ± 3 | 1252 ± 132 | 16 ± 1.3 | 193 ± 114 | 86 ± 61 | 70 ± 21 | 20 ± 5 |
| LF-ApoB | 0 | 0 | 608 ± 60 | 292 ± 19 | 282 ± 65 | 30 ± 3.7 | 4417 ± 1093 | 1253 ± 142 | 3957 ± 610 | 30 ± 3 |
| (3 mpk) | | | | | | | | | | |

| | | | | | | | |
|---|---|---|---|---|---|---|---|
| LF-ApoB | 3 | 4 | ND | 12348 ± 2840 | 4938 ± 568 | 8081 ± 1268 | 54 ± 5 |
| (9 mpk) | | | | | | | |

Rats (n = 4) were IV dosed with PBS or LF01-ApoB at 3 or 9 mg/kg and then monitored for lethality and toxicities as described in FIG. 1c-d. The data are presented as mean ± SEM. ND: not done.

SUPPLEMENTARY TABLE 2
Suppression of LF1-SSB-induced toxicities by CP-690550 in rats.

| | AST | ALT | Platelets | aPTT | Cytokines (pg/ml) | | | |
|---|---|---|---|---|---|---|---|---|
| | (U/ml) | (U/ml) | (K/μl) | (sec) | IFNγ | IL-6 | MCP-1 | TNFα |
| PBS | 105 ± 5 | 39 ± 1 | 1287 ± 237 | 14.1 ± 0.5 | 215 ± 45 | 348 ± 67 | 166 ± 19 | <24 |
| LF1-SSB | 1860 ± 467 | 365 ± 19 | 163 ± 55 | 29.9 ± 0.2 | 17603 ± 3454 | 1568 ± 237 | 5051 ± 634 | 56 ± 5 |
| (3 mpk) | | | | | | | | |
| LF1-SSB + | 397 ± 12 | 104 ± 4 | 250 ± 82 | 24.3 ± 2.3 | 767 ± 351 | 175 ± 42.2 | 4192 ± 912 | 43 ± 3 |
| CP690550 | | | | | | | | |

Rats (n = 5) were dosed with PBS or CP-690550, 1 hr prior to IV administration of PBS or FL01-SSB at 3 mg/kg. Animals were monitored for lethality and red urine and blood and tissue samples were collected for various analyses as described in FIG. 3. No unscheduled death was detected in this experiment. The data are presented as mean ± SEM.

SUPPLEMENTARY TABLE 3
Activities of FK506, Etoricoxib and Aminoguanidine (AG) in suppression of LF1-SSB-induced toxicities.
A

| | AST | ALT | Platelets | aPTT | Cytokines (pg/ml) | | | |
|---|---|---|---|---|---|---|---|---|
| | (μ/L) | (μ/L) | (K/μl) | (sec) | IFNγ | IL-6 | MCP-1 | TNFα |
| PBS | 170 ± 8 | 32 ± 2 | 1326 ± 94 | 16.8 ± 1.1 | 105 ± 32 | <24 | 312 ± 76 | <24 |
| LF-SSB | 818 ± 174 | 181 ± 43 | 322 ± 148 | 27.6 ± 3.1 | 17654 ± 2255 | 3156 ± 1181 | 6429 ± 910 | 74 ± 8 |
| (3 mpk) | | | | | | | | |
| LF-SSB + | 874 ± 307 | 169 ± 45 | 306 ± 108 | ND | 22650 ± 1957 | 2146 ± 594 | 4689 ± 906 | 62 ± 7 |
| FK506 | | | | | | | | |
| LF-SSB + | 881 ± 341 | 393 ± 199.3 | 174 ± 3 | 27.1 ± 2.5 | 10683 ± 1113 | 5791 ± 1287 | 4646 ± 448 | 78 ± 14 |
| Etoricoxib | | | | | | | | |

Rats (n = 5) were dosed with PBS, FK506 or etoricoxib 1 hr prior to IV administration of PBS or FL01-SSB at 3 mg/kg.
Blood and tissue samples were collected for various analyses as described in FIG. 3. No unscheduled death was detected
in this experiment. The data are presented as mean ± SEM.

B

| AST | ALT | Platelets | aPTT | Cytokines (pg/ml) | | | |
|---|---|---|---|---|---|---|---|
| (μ/L) | (μ/L) | (K/μl) | (sec) | IFNγ | IL-6 | MCP-1 | TNFα |

| | | | | |
|---|---|---|---|---|
| PBS | 162 ± 6 | 66 ± 3 | 1232 ± 168 | ND |
| LF-SSB | 1308 ± 851 | 320 ± 195 | 511 ± 103 | |
| (3 mpk) | | | | |
| LF-SSB + | 784 ± 134 | 187 ± 32 | 482 ± 84 | |
| AG | | | | |

Rats (n = 4) were dosed with PBS or aminoguanidine (100 mg/kg), 30 min prior to IV administration of PBS or FL01-SSB
at 3 mg/kg. Blood was collected for serum chemistry an CBC analyses 16 hr post FL01-SSB dose. The data are presented as
mean ± SEM. ND: not done

### DISCUSSION

Whereas liposome-based delivery vehicles were effective in delivering siRNA to hepatocytes^{7, 8, 10, 30}, the toxicities associated with liposomal siRNA nanoparticles limit the therapeutic window and represent a major challenge for the development of liposomal siRNA as a new modality of therapeutics^{4, 11}. As shown in this report, liposomal siRNA may induce multi-systemic toxicities and even lethality despite the effort to use chemically modified siRNAs.

Therefore, an in-depth understanding of the etiology of liposomal siRNA-induced pathologies is essential for identifying the prevention and mitigation strategies and for designing assays to select for safer liposomal formulations. Four types of toxicities were observed in rats following systemic administration of LF01-siRNA nanoparticles, namely 1) induction of multiple proinflammatory cytokines, 2) hepatic, splenic and renal impairments, manifested by an elevation of serum ALT and AST, cell death in the liver and spleen, and visible hematuria, 3) thrombocytopenia and 4) coagulopathy (FIGS. 1, 3, 4). Since LF01 formulated with distinct siRNA sequences targeting different genes (SSB and ApoB) caused comparable toxic responses (FIG. 1 and Supplementary Table 1), it is unlikely that the observed toxicities are caused by a specific siRNA sequence or due to the repression of a specific gene. Recapitulation of similar toxic responses in mice (data not shown) suggests that LF01-siRNA-associated toxicities are across species. LF01-siRNA nanoparticles may induce multi-systemic toxicities in two different modes. First, they may trigger one initial toxic event which in turn elicits subsequent toxicities involving different systems. Second, they may cause multifaceted toxicities independently by interacting with the components of different systems such as immune cells, platelets, endothelial cells, hepatocytes and plasma proteins, etc. In the former mode, blocking the initial toxic event can prevent secondary pathological responses and thus identification of the triggering toxic event and the underlying mechanism is crucial. Since activation of the innate immune response characterized by robust induction of multiple cytokines is commonly seen among liposomal siRNA-induced toxicities and it occurs at an early stage of toxic responses, we determined the activities of both multifunctional and pathway-specific suppressors of the innate immune response and inflammation in the suppression of LF01-siRNA toxicities. Our finding that Jak2 inhibitors and dexamethasone can block LF01-siRNA-induced lethality and multi-systemic toxicities discloses that LF01-siRNA-induced toxic responses are sequential and interdependent and that activation of the innate immune response is a primary trigger (FIGS. 1, 3, 4 and Table 1). This observation is consistent with former demonstrations that over-production of cytokines could damage multiple organs, disrupt hematopoietic homeostasis and cause coagulation disorders, and that overstimulation of the immune system induced multi-systemic toxicities in clinical trials^{11, 12, 28, 29, 48, 49}.

While the suppression of LF01-siRNA-triggered innate immune response by dexamethasone is expected due to its ability in inhibiting multiple pathways of innate immunity and in blocking inflammation^{32, 33}, the differential activities in mitigating cytokine response and other toxicities by pathways-specific inhibitors are intriguing and shed light on the pathways required for mediating LF01-siRNA immunotoxicity (FIGS. 3, 4, 5, Table 1, Supplementary Table 3). Stimulation of TLRs and/or cytoplasmic immunoreceptors, such as RIG-1 and MDA-5, can activate multiple downstream pathways such as NFκB, AP1, PI3K and IRF3/5/7 which lead to the induction of cytokines and chemokines^{17, 18, 20-24}. Partial, but not complete, mitigation of cytokine induction and other toxicities by wortmannin, rapamycin, p38-I and IKK1/2-I which inhibit PI3K, mTOR (a component of the PI3K pathway), API, and NFκB pathways respectively, suggests the functional overlap of these pathways in mediating cytokine induction upon stimulation by LF01-siRNA. In addition, although wortmannin and p38-I abrogated the induction of IFNγ and IL-6 respectively (FIG. 5a), neither of these agents was able to completely rescue LF01-siRNA-induced lethality and toxicities (Table 1 and FIG. 5), This suggests that suppression of a single cytokine is not sufficient to eliminate cytokine-mediated pathological consequences. In contrast to the roles of PI3K, p38 and IKK1/2 in the innate immune system, Jak2 associates with the receptors of a group of cytokines including IFNγ, IL-6, IL-3, GM-CSF, G-CSF and erythropoietin, etc. and it is required for mediating the functionality of these cytokines in terms of amplifying cytokine production, executing inflammatory responses and stimulating the growth of immune cells and erythrocytes^{25, 26}. A complete blockade of LF01-siRNA-triggered lethality and systemic toxicities by Jak2 inhibitors indicates that the Jak2-dependent cytokine response is essential for inducing secondary toxic responses. Among cytokines whose response is coupled with Jak2, IFNγ and IL-6 belong to the most robustly induced cytokines in rats following exposure to LF01-siRNA. A profound inhibition of IFNγ, IL-6 and MCP-1 and a moderate suppression of TNFα by Jak2-IA suggest that a full induction of these cytokines is Jak2-dependent and that these cytokines are important candidates for triggering subsequent toxicities. COX2 and iNOS are downstream effectors of inflammatory response regulated by cytokines^{35, 36}, while NFAT participates in T cell activation³⁴. The lack of protective activities of etoricoxib, aminoguanidine and FK506 that inhibit COX2, iNOS and NFAT respectively reveals that none of these effectors is essential for executing toxic response triggered by LF01-siRNA. Taken together, our results suggest that induction of a group of cytokines, most likely IFNγ, IL-6, MCP-1 and TNFα, is an apical toxic event which is responsible for inducing multiple pathological consequences. Jak2 inhibitors can block the full production of these cytokine as well as IFNγ- and IL-6-mediated inflammatory reactions, thereby preventing all subsequent toxic responses. How LF01-siRNA activates the innate immune system or which TLR(s) are stimulated by LF01-siRNA is unclear and remains to be investigated.

This study provides useful guidance to the development of liposomal siRNA therapeutics. Since the induction of a group of cytokines is a primary trigger of systemic toxicities, monitoring the induction of these cytokines, not a single one, can be used for predicting the toxicities of liposomal siRNA. In the clinic, pre-medication to suppress the immune response may be a viable strategy to alleviate liposomal siRNA-associated side effects and Jak2 inhibitors can be used to prevent liposomal siRNA-induced toxicities.

### Methods

Reagents. Jak2-IA and etoricoxib (Merck & Co., Inc.) were dissolved in 10% Tween80 (vol/vol in phospate-buffered saline, PBS) and DMSO respectively for p.o. administration. Dexamethasone (Phoenix Pharmaceuticals), CP-690550 (Axon MedChem), wortmannin (Calbiochem), SB203580 (Axon MedChem), PDTC (Sigma), rapamycin (EMD Biosciences, Inc.), FK506 (Sigma) and aminoguanidine (Sigma) were formulated and administrated according to manufacturers' recommendations and the results from former studies. Dosing regiments for these reagents are listed in Table 1.

Chemically modified siRNAs including 2′-F pyrimidine, 2′-OMe or deoxy purines at ribose and inverted abasic end caps at the passenger strand as described³⁰ were synthesized at Merck & Co., Inc. The guide (antisense) strand sequences of SSB and ApoB siRNAs are as follows:
SSB: 5′-UUACAUUAAAGUCUGUUGUUU-3′ (SEQ. ID. NO.: 1); and
ApoB: 5′-AUUUCAGGAAUUGUUAAAGUU-3′ (SEQ. ID. NO.: 2).
   siRNAs were encapsulated into liposomes to produce LF01-siRNA nanoparticles by mixing the lipid mixture in an ethanol solution with an aqueous solution of siRNA at a rate of 40 ml/min through a 1 mm mixing tee, followed by stepwise diafiltration. Particle size was measured by dynamic light scattering using a Zetasizer (Malvern Instruments) and surface charge was assessed by measuring Zeta potential using ZetaPlus (Brookhaven). siRNA encapsulation efficiency was determined using a RiboGreen assay (Invitrogen). The potential endotoxin contamination was examined using a chromogenic limulus amebocyte lysate (LAL) assay (Lonza) and in all liposomal siRNA preparations used in our animal studies, the endotoxin levels at the highest dose of LF01-siRNA were <0.25 EU/kg (body weight), significantly below the endotoxin release limit for humans (5 EU/kg), defined by US Federal Drug Administration (FDA) and World Health Organization (WHO).

Rat studies. All animal studies were conducted at AAALAC-accredited Merck Research laboratories' animal facility located at West Point, Pa., and all study protocols were approved by Merck West Point Institutional Animal Care and Use Committee (IACUC). Female Sprague-Dawley (SD) rats obtained from Charles River were used in these studies at an age of 4-6 weeks and with a body weight of 120-160 grams. Anti-inflammation agents were administrated using the dosing regimens listed in Table 1, 1 hr prior to tail vein injection of liposomal siRNA nanoparticles in PBS in a volume of 0.8 ml under normal pressure. At 3 hr post injection of liposomal siRNA, ˜0.2 ml blood was collected by retro-orbital bleed under anesthesia and processed as plasma for the assessment of cytokines. At 24 hr, blood was collected for evaluation of complete blood cell counts (CBC), coagulation parameters and serum chemistry by venipuncture under anesthesia followed by exsanguination, and tissues from liver, spleen and kidney were collected for determination of SSB and ApoB mRNA levels and TUNEL analysis. For visual examination of urine color, rats were housed in metabolic cages to collect urine over a course of 24 hours after injection of liposomal siRNA nanoparticles. Animals were examined 3 times a day for detection of lethality over a course of 96 hours.

Analyses of rat blood samples. Cytokines in plasma were quantified using a microsphere bead-based, multiplexed assay, Milliplex™ RCYTO-80K-PMX23 (Millipore) which allows simultaneous quantification of 23 rat cytokines including IFNγ, IL-6, MCP-1 and TNFα. All CBC, coagulation and serum chemistry parameters were analyzed by Merck Laboratory of Animal Resources and the Safety Assessment Department at West Point, Pa. CBC was determined on whole blood placed in an EDTA-treated tube using an Advia 120 Hematology Analyzer (Siemens). Coagulation parameters in citrated plasma were assessed with a Behring Coagulation System (Siemens) and serum chemistry evaluation was conducted with an Advia 1800 Clinical Chemistry Analyzer (Siemens).

TUNEL (Terminal deoxynucleotidyl transferase-mediated dUTP-biotin Nick End Labeling). TUNEL staining in tissue sections was performed using a ‘TACS™ TdT-Blue Label in Situ Apoptosis Detection Kit’ (Trevigen). Briefly, 5 μm paraffin embedded sections of liver tissue were deparaffinized, fixed, labeled, and counterstained according to the manufacturer's recommendations. In Situ labeling procedure includes extension of 3′ ends with Biotin-dNTP (TdT Labeling Rxn), followed by additions of Strep-HRP and “TACS-Blue” Label or DAB for color development. TUNEL signal was quantified using the Arial system (Applied Imaging). Quantification of mRNA. qRT-PCR assays were used to quantify SSB and ApoB mRNA levels relative to the housekeeping gene Ppib in lysates prepared from tissues using kits from Applied Biosystems. The catalog numbers are Rn0057621_g1 for SSB, Rn01499054_m1 for ApoB and Rn03302274_m1 for Ppib.

### REFERENCES

1. Dorsett, Y. & Tuschl, T. siRNAs: applications in functional genomics and potential as therapeutics. Nat Rev Drug Discov 3, 318-329 (2004).
2. Hannon, G. J. RNA interference. Nature 418, 244-251 (2002).
3. Dykxhoorn, D. M. & Lieberman, J. Knocking down disease with siRNAs. Cell 126, 231-235 (2006).
4. Sepp-Lorenzino, L. & Ruddy, M. Challenges and opportunities for local and systemic delivery of siRNA and antisense oligonucleotides. Clin Pharmacol Ther 84, 628-632 (2008).
5. Whitehead, K. A., Langer, R. & Anderson, D. G. Knocking down barriers: advances in siRNA delivery. Nat Rev Drug Discov 8, 129-138 (2009).
6. de Fougerolles, A., Vornlocher, H. P., Maraganore, J. & Lieberman, J. Interfering with disease: a progress report on siRNA-based therapeutics. Nat Rev Drug Discov 6, 443-453 (2007).
7. Zimmermann, T. S., et al. RNAi-mediated gene silencing in non-human primates. Nature 441, 111-114 (2006).
8. Soutschek, J., et al. Therapeutic silencing of an endogenous gene by systemic administration of modified siRNAs. Nature 432, 173-178 (2004).
9. Song, E., et al. Antibody mediated in vivo delivery of small interfering RNAs via cell-surface receptors. Nat Biotechnol 23, 709-717 (2005).
10. Frank-Kamenetsky, M., et al. Therapeutic RNAi targeting PCSK9 acutely lowers plasma cholesterol in rodents and LDL cholesterol in nonhuman primates. Proc Natl Acad Sci U S A 105, 11915-11920 (2008).
11. Judge, A. & MacLachlan, I. Overcoming the innate immune response to small interfering RNA. Hum Gene Ther 19, 111-124 (2008).
12. Tousignant, J. D., et al. Comprehensive analysis of the acute toxicities induced by systemic administration of cationic lipid:plasmid DNA complexes in mice. Hum Gene Ther 11, 2493-2513 (2000).
13. Rudin, C. M., et al. Delivery of a liposomal c-raf-1 antisense oligonucleotide by weekly bolus dosing in patients with advanced solid tumors: a phase I study. Clin Cancer Res 10, 7244-7251 (2004).
14. Judge, A. D., et al. Sequence-dependent stimulation of the mammalian innate immune response by synthetic siRNA. Nat Biotechnol 23, 457-462 (2005).
15. Sioud, M. Induction of inflammatory cytokines and interferon responses by double-stranded and single-stranded siRNAs is sequence-dependent and requires endosomal localization. J Mol Blot 348, 1079-1090 (2005).
16. Kim, J. Y., Choung, S., Lee, E. J., Kim, Y. J. & Choi, Y. C. Immune activation by siRNA/liposome complexes in mice is sequence-independent: lack of a role for Toll-like receptor 3 signaling. Mol Cells 24, 247-254 (2007).
17. Schlee, M., Hornung, V. & Hartmann, G. siRNA and is RNA: two edges of one sword. Mol Ther 14, 463-470 (2006).
18. Akira, S. & Takeda, K. Toll-like receptor signalling. Nat Rev Immunol 4, 499-511 (2004).
19. Staros, E. B. Innate immunity: New approaches to understanding its clinical significance. Am J Clin Pathol 123, 305-312 (2005).
20. Barton, G. M. A calculated response: control of inflammation by the innate immune system. J Clin Invest 118, 413-420 (2008).
21. Tak, P. P. & Firestein, G. S. NF-kappaB: a key role in inflammatory diseases. J Clin Invest 107, 7-11 (2001).
22. Adams, J. L., Badger, A. M., Kumar, S. & Lee, J. C. p38 MAP kinase: molecular target for the inhibition of pro-inflammatory cytokines. Prog Med Chem 38, 1-60 (2001).
23. Rommel, C., Camps, M. & Ji, H. PI3K delta and PI3K gamma: partners in crime in inflammation in rheumatoid arthritis and beyond? Nat Rev Immunol 7, 191-201 (2007).
24. Guiducci, C., et al. PI3K is critical for the nuclear translocation of IRF-7 and type I IFN production by human plasmacytoid predendritic cells in response to TLR activation. J Exp Med 205, 315-322 (2008).
25. Ihle, J. N. Cytokine receptor signalling. Nature 377, 591-594 (1995).
26. Imada, K. & Leonard, W. J. The Jak-STAT pathway. Mol Immunol 37, 1-11 (2000).
27. Karin, M., Yamamoto, Y. & Wang, Q. M. The IKK NF-kappa B system: a treasure trove for drug development. Nat Rev Drug Discov 3, 17-26 (2004).
28. Suntharalingam, G., et al. Cytokine storm in a phase 1 trial of the anti-CD28 monoclonal antibody TGN1412. N Engl J Med 355, 1018-1028 (2006).
29. Raper, S. E., et al. Fatal systemic inflammatory response syndrome in a ornithine transcarbamylase deficient patient following adenoviral gene transfer. Mol Genet Metab 80, 148-158 (2003).
30. Morrissey, D. V., et al. Potent and persistent in vivo anti-HBV activity of chemically modified siRNAs. Nat Biotechnol 23, 1002-1007 (2005).
31. Park, J. M., et al. The multifunctional RNA-binding protein La is required for mouse development and for the establishment of embryonic stem cells. Mol Cell Biol 26, 1445-1451 (2006).
32. Moynagh, P.N. Toll-like receptor signalling pathways as key targets for mediating the anti-inflammatory and immunosuppressive effects of glucocorticoids. J Endocrinol 179, 139-144 (2003).
33. Morand, E. F. Effects of glucocorticoids on inflammation and arthritis. Curr Opin Rheumatol 19, 302-307 (2007).
34. Magari, K., Miyata, S., Ohkubo, Y., Mutoh, S. & Goto, T. Calcineurin inhibitors exert rapid reduction of inflammatory pain in rat adjuvant-induced arthritis. Br J Pharmacol 139, 927-934 (2003).
35. Riendeau, D., et al. Etoricoxib (MK-0663): preclinical profile and comparison with other agents that selectively inhibit cyclooxygenase-2. J Pharmacol Exp Ther 296, 558-566 (2001).
36. Lowenstein, C. J. & Padalko, E. iNOS(NOS2) at a glance. J Cell Sci 117, 2865-2867 (2004).
37. Hermens, W. T. & Verhaagen, J. Suppression of inflammation by dexamethasone prolongs adenoviral vector-mediated transgene expression in the facial nucleus of the rat. Brain Res Bull 47, 133-140 (1998).
38. Seregin, S. S., et al. Transient pretreatment with glucocorticoid ablates innate toxicity of systemically delivered adenoviral vectors without reducing efficacy. Mol Ther 17, 685-696 (2009).
39. Kudlacz, E., et al. The novel JAK-3 inhibitor CP-690550 is a potent immunosuppressive agent in various murine models. Am J Transplant 4, 51-57 (2004).
40. Podolin, P. L., et al. Attenuation of murine collagen-induced arthritis by a novel, potent, selective small molecule inhibitor of IkappaB Kinase 2, TPCA-1 (2-[(aminocarbonyl)amino]-5-(4-fluorophenyl)-3-thiophenecarboxamide), occurs via reduction of proinflammatory cytokines and antigen-induced T cell Proliferation. J Pharmacol Exp Ther 312, 373-381 (2005).
41. Badger, A. M., et al. Pharmacological profile of SB 203580, a selective inhibitor of cytokine suppressive binding protein/p38 kinase, in animal models of arthritis, bone resorption, endotoxin shock and immune function. J Pharmacol Exp Ther 279, 1453-1461 (1996).
42. Adams, J. L., et al. Pyrimidinylimidazole inhibitors of p38: cyclic N-1 imidazole substituents enhance p38 kinase inhibition and oral activity. Bioorg Med Chem Lett 11, 2867-2870 (2001).
43. Yatscoff, R. W., Wang, P., Chan, K., Hicks, D. & Zimmerman, J. Rapamycin: distribution, pharmacokinetics, and therapeutic range investigations. Ther Drug Monit 17, 666-671 (1995).
44. Wu, C.C., Chen, S. J., Szabo, C., Thiemermann, C. & Vane, J. R. Aminoguanidine attenuates the delayed circulatory failure and improves survival in rodent models of endotoxic shock. Br J Pharmacol 114, 1666-1672 (1995).
45. Changelian, P. S., et al. Prevention of organ allograft rejection by a specific Janus kinase 3 inhibitor. Science 302, 875-878 (2003).
46. Ui, M., Okada, T., Hazeki, K. & Hazeki, O. Wortmannin as a unique probe for an intracellular signalling protein, phosphoinositide 3-kinase. Trends Biochem Sci 20, 303-307 (1995).
47. Schindler, C. Cytokines and JAK-STAT signaling. Exp Cell Res 253, 7-14 (1999).
48. Joseph, L., Fink, L. M. & Hauer-Jensen, M. Cytokines in coagulation and thrombosis: a preclinical and clinical review. Blood Coagul Fibrinolysis 13, 105-116 (2002).
49. Furie, M. B. & Randolph, G. J. Chemokines and tissue injury. Am J Pathol 146, 1287-1301 (1995).

What is claimed is:

1. A method for treating a patient, wherein the patient will be or is currently being treated with a lipid-based nucleic acid therapeutic, by administering a JAK inhibitor.

2. The method of claim 1 wherein the JAK inhibitor is a JAK2 inhibitor.

3. The method of claim 1 wherein the JAK inhibitor is selected from Jak2-IA, AG490, Pyridone 6, WP1066, LS104, TG101209, TG101348, CP690,550, CP352,664, INCB18424, WHI-P154, CMP6, SB1518, XL019, CEP-701, INCB20, AUH-6-96 and AZ960.

4. The method of claim 1 wherein the patient is pre-treated with a JAK inhibitor.

5. The method of claim 1 wherein the patient is co-treated with a JAK inhibitor.


SEQUENCE LISTING (2 sequences)

SEQ ID NO: 1 - RNA, 21 nt, Artificial Sequence
  Enzymatic Nucleic Acid
uuacauuaaagucuguuguuu

SEQ ID NO: 2 - RNA, 21 nt, Artificial Sequence
  Enzymatic Nucleic Acid
auuucaggaauuguuaaaguu